(19) United States
(12) Patent Application Publication
(10) Pub. No.: US 2005/0249968 A1
(43) Pub. Date: Nov. 10, 2005

(54) Whisker inhibition in tin surfaces of electronic components

(75) Inventors: Chen Xu, New Providence, NJ (US); Yun Zhang, Warren, NJ (US); Chonglun Fan, Bridgewater, NJ (US); Oscar Khaselev, Monmouth Junction, NJ (US); Joseph A. Abys, Warren, NJ (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Enthone Inc.

(21) Appl. No.: 10/838,571

(22) Filed: May 4, 2004

(51) Int. Cl. B32B 15/01; C25D 5/12

(52) U.S. Cl. 428/647; 428/648; 427/123; 205/181; 205/170

(57) ABSTRACT

A method for reducing whisker formation in tin coatings over metal features of electronic components. The tin coating has internal tensile stress and is between about 0.5 μm and about 4.0 μm in thickness.

### FIELD OF THE INVENTION

The present invention relates generally to a method for improving the integrity of tin coatings and, thereby, the performance of electronic components utilizing metal features having tin coatings. The present invention further relates to a method for inhibiting the formation of whiskers in tin coatings on metal features of electronic components. For example, components such as lead lines of lead frames, electrical connectors, and passive components such as chip capacitors and chip resistors often have tin-coated metal features.

### BACKGROUND OF THE INVENTION

For much of its history, the electronics industry has relied on tin-lead solders to make connections in electronic components. Under environmental, competitive, and marketing pressures, the industry is moving to alternative solders that do not contain lead. Pure tin is a preferred alternative solder because of the simplicity of a single metal system, tin's favorable physical properties, and its proven history as a reliable component of popular solders previously and currently used in the industry. The growth of tin whiskers is a well known but poorly understood problem with pure tin coatings. Tin whiskers may grow between a few micrometers to a few millimeters in length, which is problematic because they can electrically connect multiple features resulting in electrical shorts. The problem is particularly pronounced in high pitch input/output components with closely configured features, such as lead frames and connectors.

Electrical components are mechanically and electrically connected to larger electronic assemblies by lead lines. The integrated circuit (IC) or other discrete electrical device is mechanically mounted on a lead frame's paddle and then electrically connected to the numerous lead lines. Typically, the device is encapsulated at this point to maintain the integrity of the mechanical and electrical connections. The electronic component, comprising the device attached to the lead frame, is then electrically and mechanically connected to a larger assembly, such as a printed wiring board (PWB). Copper and copper alloys have been widely used as the base lead frame material, in part because of their mechanical strength, conductivity, and formability. But copper and its alloys do not display the requisite corrosion resistance or solderability, necessitating a coating thereover to impart these desired characteristics. A tin-lead coating has been employed to impart solderability to the copper lead frame.

In addition to lead frames, electrical connectors are an important feature of electrical components used in various applications, such as computers and other consumer electronics. Connectors provide the path whereby electrical current flows between distinct components. Like lead frames, connectors should be conductive, corrosion resistant, wear resistant, and solderable. Again, copper and its alloys have been used as the connectors' base material because of their conductivity. Thin coatings of tin have been applied to connector surfaces to assist in corrosion resistance and solderability. Tin whiskers in the tin coating present a problem of shorts between electrical contacts.

In practice, lead frames have been typically coated with tin-based coatings between about 8 to 15 μm thick, while electrical connectors are typically coated with tin-based coatings that are about 3 μm thick. Conventional wisdom has deemed such thicker coatings preferable for preventing tin whisker growth and general coating integrity.

Accordingly, a need continues to exist for electrical components with a coating that imparts corrosion resistance and solderability without a propensity for whisker growth.

### SUMMARY OF THE INVENTION

Among the objects of the invention, therefore, is the provision of a tin-based coating for electrical components, especially lead frames and electrical connectors, and passive components such as chip capacitors and chip resistors, which provides solderability and corrosion resistance and has a reduced tendency for tin whisker formation.

Briefly, therefore, the invention is directed to a method for applying a solderable, corrosion-resistant, tin-based coating having a resistance to tin whisker formation onto a metal surface of an electronic component. A first metal layer is deposited onto the metal surface, wherein the first metal layer comprises a metal or alloy which establishes a diffusion couple with the tin-based coating that promotes a bulk material deficiency in the tin-based coating and, thereby, an internal tensile stress in the tin-based coating. A thin tin-based coating is deposited over the first metal layer.

Other objects and features of this invention will be in part apparent and in part pointed out hereinafter.

### BRIEF DESCRIPTION OF THE DRAWINGS

FIG. 1 is a schematic cross section of a lead formed according to this invention for an encapsulated electronic component.

FIG. 2 is a Dual Inline Package (DIP) electronic component.

FIG. 3 is a lead frame.

FIG. 4 is an electrical connector.

FIG. 5 is a schematic of the mechanism by which tensile stress is created within the tin-based coating.

FIG. 6 is a schematic of the mechanism by which whiskers form in tin-based coatings on copper substrates.

FIGS. 7a and 7b are 1000× and 500× photomicrographs, respectively, of a 10 μm tin-based coating's surface after testing according to Example 2.

FIGS. 8a and 8b are 1000× and 500× photomicrographs, respectively, of a 3 μm tin-based coating's surface after testing according to Example 2.

FIGS. 9a and 9b are 1000× and 500× photomicrographs, respectively, of a 2 μm tin-based coating's surface after testing according to Example 2.

FIGS. 10a and 10b are 1000× and 500× photomicrographs, respectively, of a 1 μm tin-based coating's surface after testing according to Example 2.

FIGS. 11a and 11b are 1000× and 500× photomicrographs, respectively, of a 0.5 μm tin-based coating's surface after testing according to Example 2.

FIG. 12 is a graph of the Whisker Index of the five samples prepared according to Example 2.

### DETAILED DESCRIPTION OF THE PREFERRED EMBODIMENTS

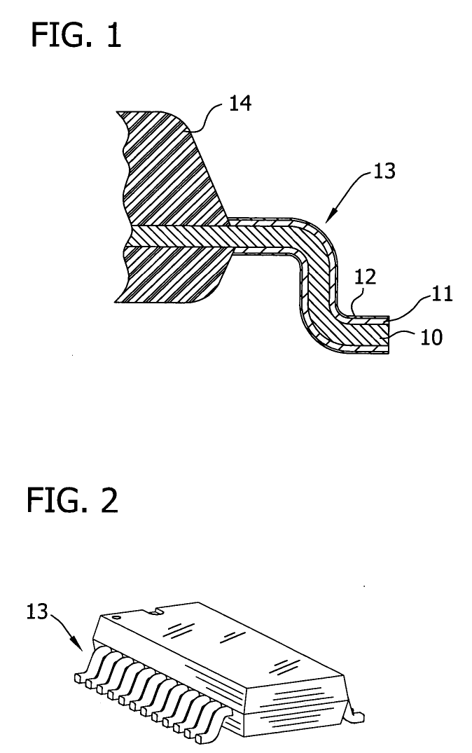

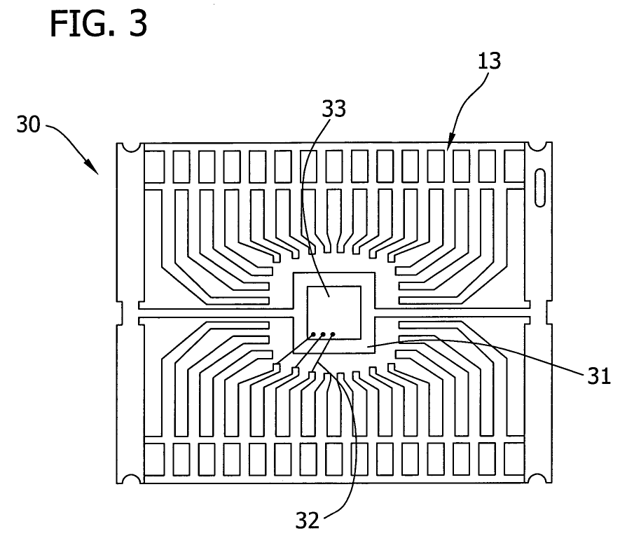

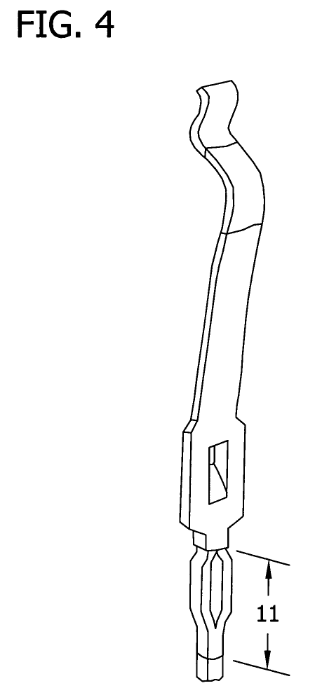

In accordance with this invention, a tin-based coating having a reduced tendency for whisker formation is formed on a metal surface of an electronic component. An electronic device can be formed by combining several electronic components. In one aspect, this invention encompasses a lead 13 as shown in FIG. 1. This lead 13 is a segment of any standard electronic package employing leads, such as the dual inline package displayed in FIG. 2, which is manufactured in part from a lead frame 30 shown in FIG. 3. In FIG. 3, the electronic device 33 is positioned on a pad 31 and connected to leads 13 by wire bonds 32. In another aspect, this invention encompasses an electronic connector as shown in FIG. 4. Referring again to FIG. 1, a cross section of part of an electronic package is shown with a lead 13 having a conductive base metal 10, a first metal layer 11 on the base metal's surface, and a tin or tin alloy coating 12. The base metal may be copper, a copper alloy, iron, an iron alloy, or any other metal suitable for use in electronic components. A tin or tin alloy coating is applied to provide corrosion resistance and solderability to the metal feature. Examples of tin alloys employed include Sn—Bi, Sn—Cu, Sn—Zn, Sn—Ag.

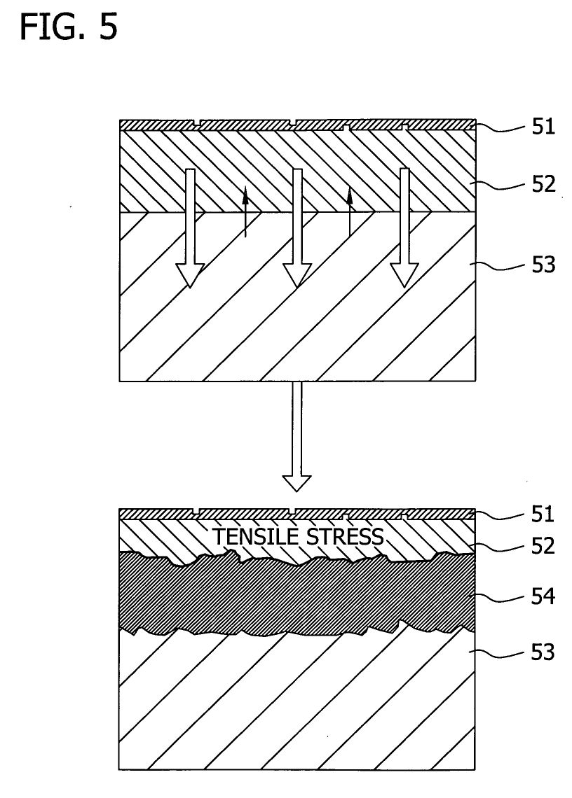

The first metal layer 11 is a metal or alloy that cooperates with the tin-based coating 12 to create a diffusion couple wherein the tin atoms from 12 diffuse more quickly into the metal layer 11 than the metal layer's atoms diffuse into the tin-based coating 12. By selecting a metal layer to create a diffusion couple with such properties, a bulk material deficiency of tin is created such that the tin coating is placed under an internal tensile stress. An example of this type of diffusion couple is illustrated in FIG. 5, where a tin-based coating 52 interacts with a first metal layer comprising nickel 53. While not to scale, the larger arrows of FIG. 5 represent the faster relative diffusion rate of atoms from the tin-based layer 52 into the first metal layer 53, whereas the smaller arrows represent the slower relative diffusion rate of atoms from the first metal layer 53 into the tin-based layer 52. In time, an intermetallic layer 54 comprising tin and the first metal layer material forms. In a diffusion couple employing a tin-based coating over a nickel first metal layer, Ni₃Sn₄ is an exemplary intermetallic compound 54. A tin oxide layer 51 forms on the exposed tin surface. Such a diffusion couple is important because the type of internal stress (i.e., compressive or tensile) in the tin coating has been determined to be the key factor in whisker growth. Specifically, tensile stress within the tin coating has been found to inhibit the growth of tin whiskers, whereas internal compressive stress in the tin coating facilitates whisker growth.

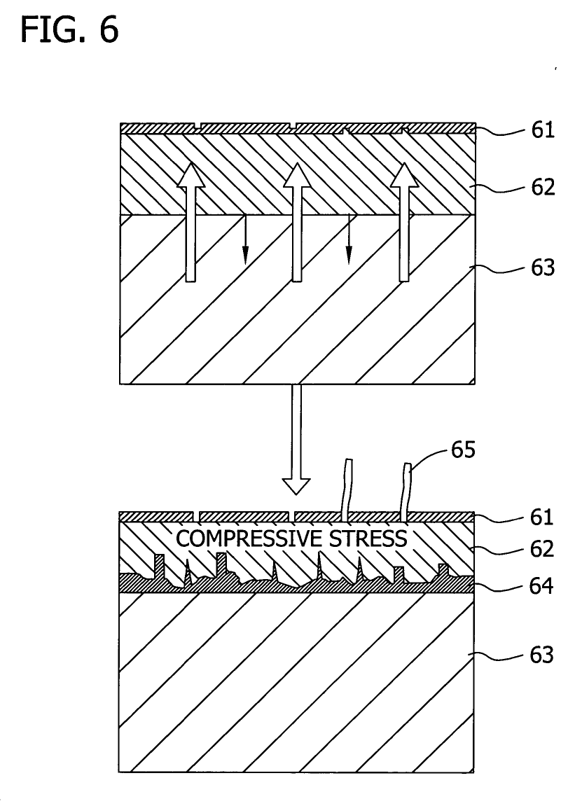

FIG. 6 shows a diffusion couple exhibiting compressive stress. Compressive stress is found in the tin-based coating 62 when tin is directly applied to a common base material 63, such as copper and its alloys, because tin atoms diffuse into the base material 63 more slowly than the base material's atoms diffuse into the tin-based coating 62. While not to scale, this behavior is illustrated in FIG. 6 by the relative size of the arrows between the tin-based layer 62 and the base material 63, eventually forming an intermetallic layer 64. The compressive stress in the tin-based layer 62 promotes the growth of tin whiskers 65 through the tin oxide layer 61. Therefore, the metal layer material is critical to the formation of a tin coating without whiskers.

Compressive stress is also introduced to the tin-based layer when the electronic component is heated, which may occur while powering the electronic component or with normal variations in the ambient temperature. When an electronic component having a tin-based coating on a metal (e.g., Cu) substrate is subjected to a temperature change, thermal stresses are created within the tin coating because there is a mismatch in the base material's coefficient of thermal expansion (CTE) vis-à-vis the tin-based coating's CTE. For tin on nickel or tin on copper, the net thermal stress is compressive in the tin coating during the heating cycle because of tin's higher linear CTE (23 μin/in-° C.) as compared to a nickel-based first metal layer (13.3 μin/in-° C. for pure nickel) or a copper-based conductive material (16.5 μin/in-° C. for pure copper). These values show that tin expands and contracts more readily than the underlying materials in response to temperature changes. The internal compressive stress created by this CTE mismatch promotes whisker formation. This invention involves controlling the magnitude of the compressive stress resulting from CTE mismatch, and establishing opposing tensile stress that is sufficient to counteract the compressive stress, thereby reducing the tendency for whisker formation.

With reference to FIG. 1, the thickness of the tin-based coating 12 is limited so that any compressive stress created in the coating is offset by the tensile stress derived from a diffusion couple. Regardless of the tin-based coating's thickness, the thermal stress from heating is compressive at all points in the Sn coating. Opposing tensile stress is imparted to a localized portion of the coating by creating a diffusion couple between the first metal layer 11 and the tin-based coating 12 that promotes a bulk material deficiency and, thereby, internal tensile stress. Since this tensile stress is localized near the diffusion couple, a thicker coating has some points of the tin-based coating where the compressive thermal stress is not influenced by the tensile stress purely because of distance therefrom. Thus, in all embodiments of the invention, the tin-based coating is sufficiently thin so that all points in its thickness experiencing compressive thermal stress are dominated by countervailing localized tensile stress from the diffusion couple.

In one preferred embodiment, the first metal layer 11 in FIG. 1 comprises nickel or a nickel alloy because nickel establishes the requisite diffusion couple with tin. That is, nickel establishes a diffusion couple with tin which promotes a bulk material deficiency and, thereby, internal tensile stress in the tin-based coating. Examples of suitable nickel alloys include Ni—Co and Ni—Fe. Other candidate underlayer materials include Co and Co alloys, Fe and Fe alloys, and Ag and Ag alloys. This first metal layer 11 in one preferred embodiment has a thickness of between about 0.1 μm and 20 μm.

The tin-based coating 12 on the lead line has a thickness at least about 0.5 μm, but less than 4.0 μm. In one embodiment, it is less than 3.0 μm. A thicker tin-based coating, such as from 4 μm to 8 μm, or even to 15 μm, as have been applied to copper lead lines with or without optional first metal layer coatings is specifically avoided. In certain preferred embodiments, the thickness is maintained at or below about 2.5 μm. In certain other preferred embodiments, the thickness is maintained at or below about 2.0 μm.

Where the substrate is an electrical connector, as shown in FIG. 4, the tin-based coating 11 on the connector has a thickness of at least about 0.5 μm, but less than about 2.5 μm. A thicker tin-based coating, such as 3 μm or greater, as has been applied to previous connectors is specifically avoided. In certain preferred embodiments, the thickness is maintained at or below about 2.0 μm. In certain other preferred embodiments, the thickness is maintained between about 0.5 and about 1.0 μm.

In carrying out the invention, the first metal layer is applied to the conductive base metal's surface, such as to the surface of the lead line 10 in FIG. 1. To this end, electrodeposition can be used to apply the first metal layer to the metal's surface. An example of suitable electrodeposition chemistry is the Sulfamex system disclosed in the below examples. Next, a tin-based coating is applied on top the first metal layer. Again, electrodeposition can be used to apply the tin-based coating to the first metal layer. An example of suitable electrodeposition chemistry is the Stannostar chemistry available from Enthone Inc. of West Haven, Conn. employing Stannostar additives (e.g., wetting agent 300, C1, C2, or others). Other methods such as PVD and CVD are possible, but electrodeposition is typically much less expensive.

For lead frames, the underlayer and Sn coating are typically applied to the exposed lead line after application of encapsulation. Here, the underlayer and Sn coating terminate where the encapsulation of the lead line begins. Less often, the underlayer and Sn coating are applied earlier in the process, i.e., to the lead frame shown in FIG. 3. This former process is shown with the schematic illustration in FIG. 1 because the underlayer 11 and Sn coating 12 do not extend under the encapsulation 14 of the lead line 10.

The present invention is illustrated by the following examples, which are merely for the purpose of illustration and not to be regarded as limiting the scope of the invention or manner in which it may be practiced.

### EXAMPLE 1

Five samples were prepared by first electrodepositing a first metal layer of conformable nickel using the Sulfamex MLS plating system, available from Enthone, Inc. of West Haven, Conn., on a C19400 copper alloy substrate. To this end, an electrolytic bath was prepared comprising the following, in deionized water:
Ni(NH₂SO₃)₂−319-383 g/L
NiCl₂*6H₂O−5-15 g/L
H₃BO₃−20-40 g/L
CH₃(CH₂)₁₁OSO₃Na−0.2-0.4 g/L

The electrolytic bath was maintained at a pH between about 2.0 and about 2.5. The bath was held at a temperature between about 55° C. and about 65° C. A current density between about 20 A/ft² and about 300 A/ft² for a time sufficient to apply a first metal layer of nickel alloy approximately 2 μm thick.

Next, a matte tin alloy coating was electrodeposited on each of the five samples using the STANNOSTAR plating system available from Enthone, Inc. To this end, an electrolytic bath was prepared comprising the following, in deionized water:
Sn(CH₃SO₃)₂−40-80 g/L
CH₃SO₃H−100-200 g/L
Stannostarr Additives−1-15 g/L

The electrolytic bath was maintained at a pH of about 0. The bath was held at a temperature of about 50° C. A current density of about 100 A/ft² was applied for a time sufficient to apply the desired coating thickness to each of the samples. Here, the samples were coated with 10 μm, 3 μm, 2 μm, 1 μm, and 0.5 μm of matte tin alloy.

### EXAMPLE 2

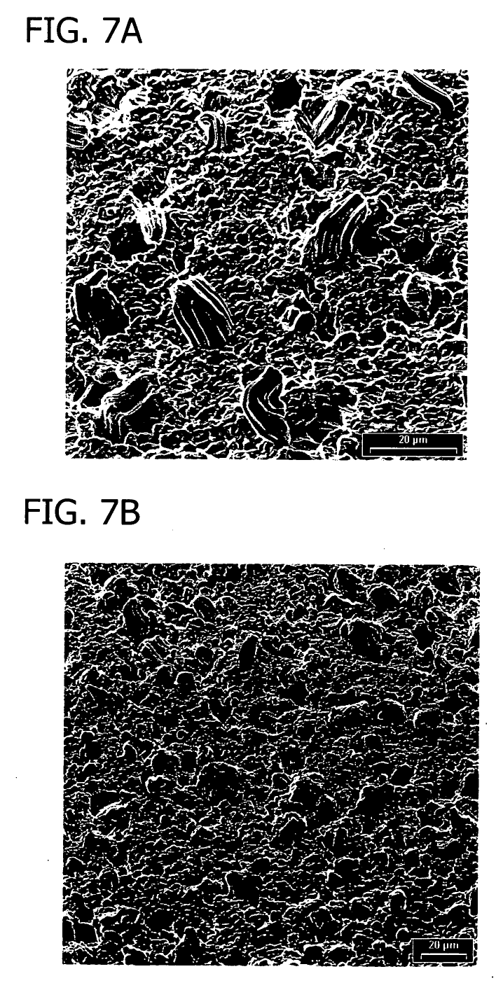

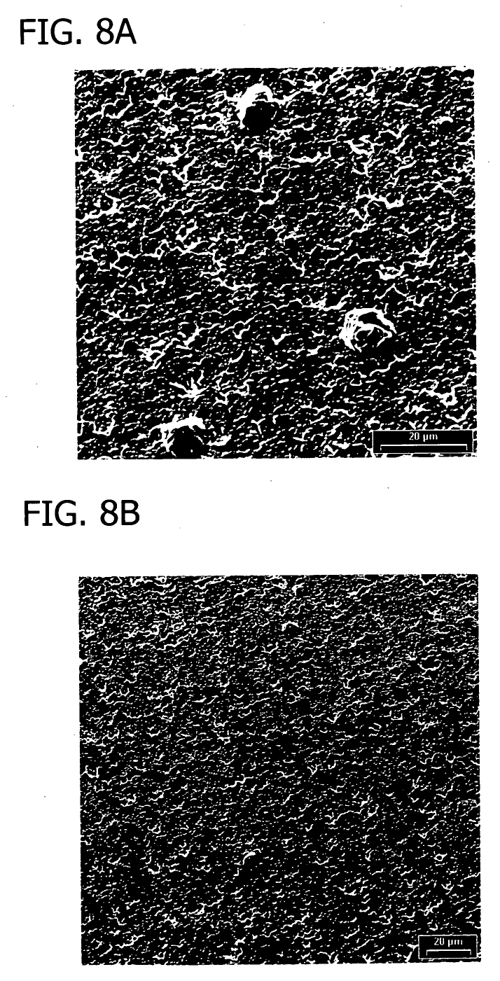

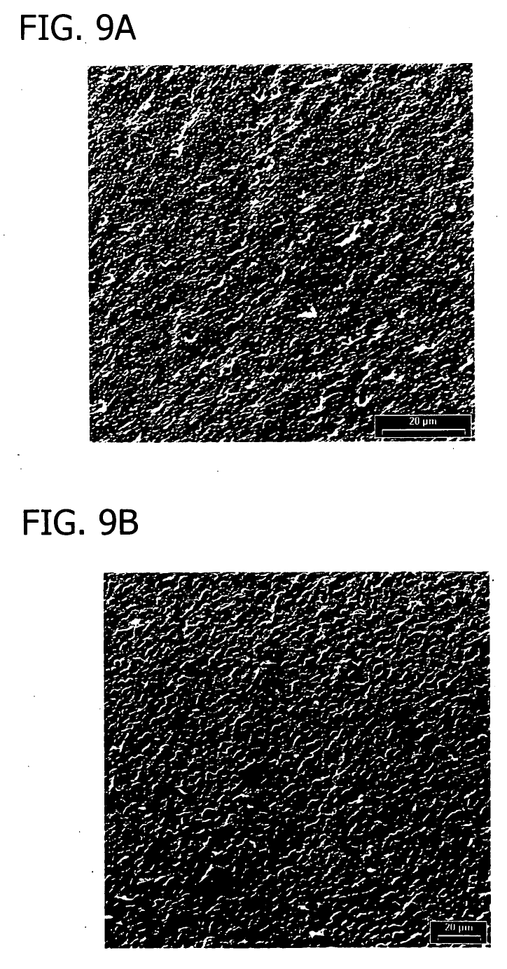

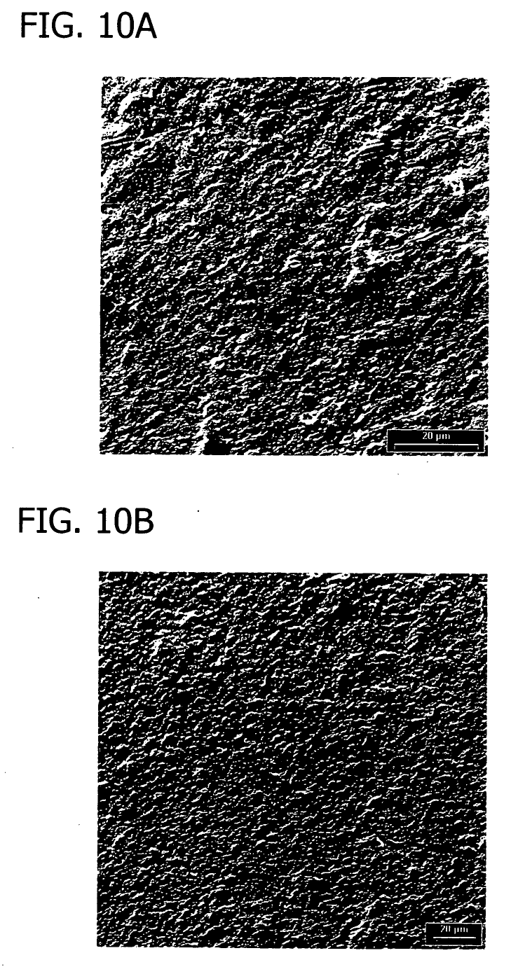

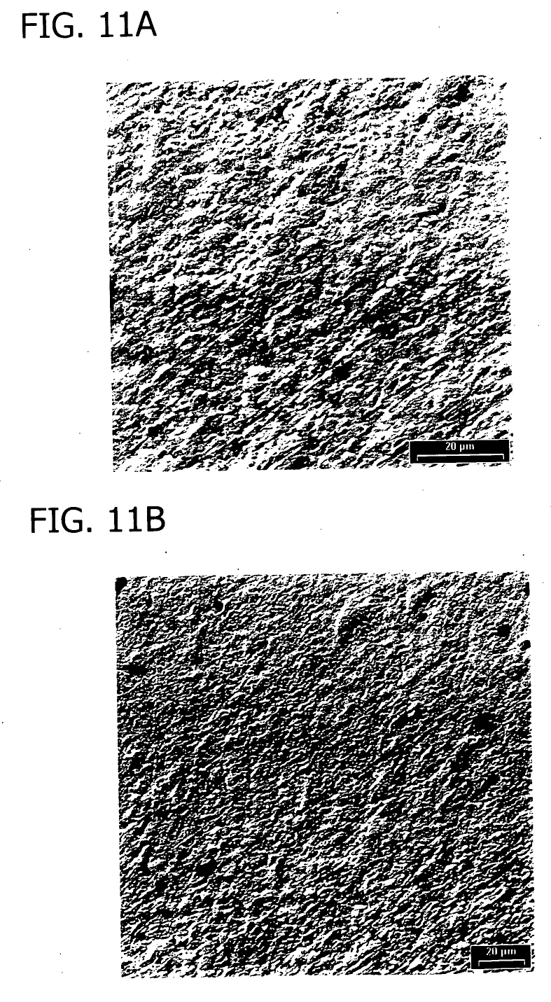

The five samples prepared according to Example 1 were subjected to 1000 thermal shock cycles from about −55° C. to about 85° C. FIGS. 7-11 are photomicrographs of the samples after this thermal shock testing. FIGS. 7a and 7b, 1000× and 500× respectively, show growth of many tin whiskers of substantial size in the sample with a 10 μm thick tin alloy coating. FIGS. 8a and 8b, 1000× and 500× respectively, show growth of a few tin whiskers of notable size in the sample with a 3 μm thick tin alloy coating. FIGS. 9a and 9b, 1000× and 500× respectively, show growth of very few tin whiskers of negligible size in the sample with a 2 μm thick tin alloy coating. FIGS. 10a and 10b, 1000× and 500× respectively, show virtually no growth of tin whiskers in the sample with a 1 μm thick tin alloy coating. Similarly, FIGS. 11a and 11b, 1000× and 500× respectively, show virtually no growth of tin whiskers in the sample with a 0.5 μm thick tin alloy coating.

### EXAMPLE 3

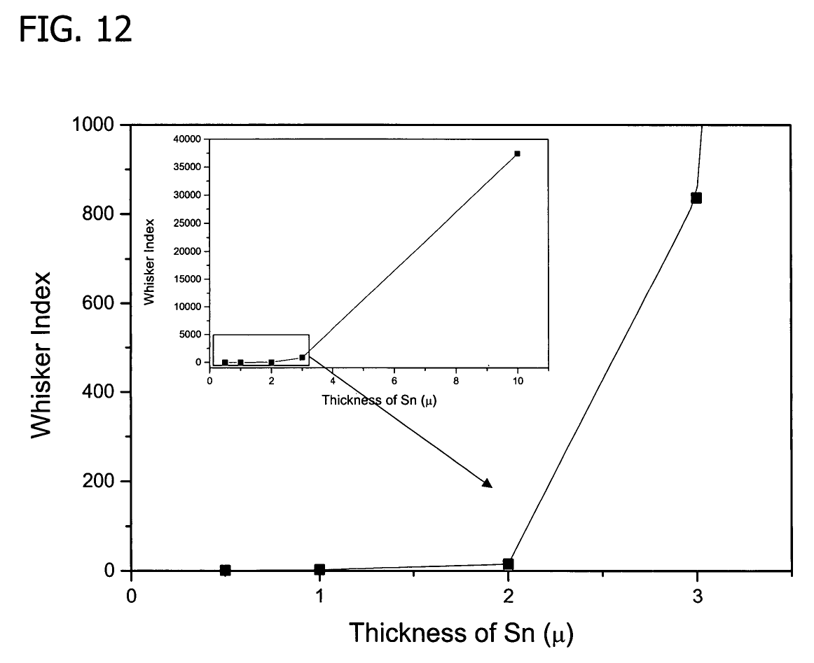

FIG. 12 shows a graph comparing the Whisker Index (WI) for each of the five samples prepared according to Example 1 after the thermal shock testing of Example 2. The WI for a tin alloy coating is a value that is defined as a function of the number of whiskers, the length of the whiskers, the diameter of the whiskers, and the “weighing factor” of the whiskers in a given area of a sample. The weighing factor helps differentiate short and long whiskers. Here, the WI for each of the five sample was determined using the 500× photomicrographs, 7b, 8b, 9b, 10b, and 11b. As indicated in FIG. 12, the WI increases dramatically from nearly 0 for the 2 μm sample to approximately 825 for the 3 μm sample, to substantially greater where the tin-based coating is above about 3 μm.

The present invention is not limited to the above embodiments and can be variously modified. The invention is not limited to leadframes and connectors, and extends to other components including passive components such as chip capacitors and chip resistors. The above description of preferred embodiments is intended only to acquaint others skilled in the art with the invention, its principles and its practical application so that others skilled in the art may adapt and apply the invention in its numerous forms, as may be best suited to the requirements of a particular use.

With reference to the use of the word(s) “comprise” or “comprises” or “comprising” in this entire specification (including the claims below), it is noted that unless the context requires otherwise, those words are used on the basis and clear understanding that they are to be interpreted inclusively, rather than exclusively, and that it is intended each of those words to be so interpreted in construing this entire specification.

1. A method for applying a solderable, corrosion-resistant, tin-based coating having a resistance to tin whisker formation onto a metal surface of an electronic component, the method comprising:
depositing a first metal layer onto the metal surface, wherein the first metal layer comprises a metal or alloy which establishes a diffusion couple with the tin-based coating that promotes a bulk material deficiency in the tin-based coating and, thereby, an internal tensile stress in the tin-based coating; and
depositing the tin-based coating over the first metal layer to a thickness between about 0.5 μm and about 2.5 μm.

2. The method of claim 1 wherein the first metal layer comprises nickel or a nickel alloy.

3. The method of claim 1 wherein the metal surface of the electronic component is a metal selected from the group consisting of copper, copper alloys, iron, and iron alloys.

4. The method of claim 1 wherein the first metal layer has a thickness between about 0.1 μm and about 20 μm.

5. The method of claim 1 wherein the electronic component is a lead line of an electronic package for incorporation into an electronic device.

6. The method of claim 1 wherein the electronic component is a lead line of an electronic package for incorporation into an electronic device, and the method comprises:
depositing the first metal layer onto the metal surface of the lead line, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.5 μm.

7. The method of claim 1 wherein the electronic component is a lead line of an electronic package for incorporation into an electronic device, and the method comprises:
depositing the first metal layer onto the metal surface of the lead line, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.0 μm.

8. The method of claim 1 wherein the electronic component is a lead line of an electronic package for incorporation into an electronic device, and the method comprises:
depositing the first metal layer onto the metal surface of the lead line, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.5 μm.

9. The method of claim 1 wherein the electronic component is a lead line of an electronic package for incorporation into an electronic device, and the method comprises:
depositing the first metal layer onto the metal surface of the lead line, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.0 μm.

10. The method of claim 1 wherein the electronic component is a lead line of an electronic package for incorporation into an electronic device, and the method comprises:
depositing the first metal layer by electrodeposition onto the metal surface of the lead line, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.5 μm.

11. The method of claim 1 wherein the electronic component is a lead line of an electronic package for incorporation into an electronic device, and the method comprises:
depositing the first metal layer by electrodeposition onto the metal surface of the lead line, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.0 μm.

12. The method of claim 1 wherein the electronic component is a lead line of an electronic package for incorporation into an electronic device, and the method comprises:
depositing the first metal layer by electrodeposition onto the metal surface of the lead line, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.5 μm.

13. The method of claim 1 wherein the electronic component is a lead line of an electronic package for incorporation into an electronic device, and the method comprises:
depositing the first metal layer by electrodeposition onto the metal surface of the lead line, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.0 μm.

14. The method of claim 1 wherein the electronic component is an electrical connector, and the method comprises:
depositing the first metal layer onto the metal surface of the electrical connector, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.5 μm.

15. The method of claim 1 wherein the electronic component is an electrical connector, and the method comprises:
depositing the first metal layer onto the metal surface of the electrical connector, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.0 μm.

16. The method of claim 1 wherein the electronic component is an electrical connector, and the method comprises:
depositing the first metal layer onto the metal surface of the electrical connector, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.5 μm.

17. The method of claim 1 wherein the electronic component is an electrical connector, and the method comprises:
depositing the first metal layer onto the metal surface of the electrical connector, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating over the first metal layer to the thickness between about 0.5 μm and about 2.0 μm.

18. The method of claim 1 wherein the electronic component is an electrical connector, and the method comprises:
depositing the first metal layer by electrodeposition onto the metal surface of the electrical connector, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.5 μm.

19. The method of claim 1 wherein the electronic component is an electrical connector, and the method comprises:
depositing the first metal layer by electrodeposition onto the metal surface of the electrical connector, wherein the first metal layer comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.0 μm.

20. The method of claim 1 wherein the electronic component is an electrical connector, and the method comprises:
depositing the first metal layer by electrodeposition onto the metal surface of the electrical connector, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.5 μm.

21. The method of claim 1 wherein the electronic component is an electrical connector, and the method comprises:
depositing the first metal layer by electrodeposition onto the metal surface of the electrical connector, wherein the first metal layer has a thickness between about 0.1 and about 20 μm and comprises Ni or Ni alloy which establishes said diffusion couple with the tin-based coating that promotes said bulk material deficiency in the tin-based coating and, thereby, said internal tensile stress in the tin-based coating; and
depositing the tin-based coating by electrodeposition over the first metal layer to the thickness between about 0.5 μm and about 2.0 μm.

22. The method of claim 1 wherein the electronic component is a passive electronic device.

23. The method of claim 22 wherein the electronic component is a chip capacitor or a chip resistor.

24. A method for applying a solderable, corrosion-resistant, tin-based coating having a resistance to tin whisker formation onto a metal lead line for an electronic package, the method comprising:
depositing a first metal layer onto the metal lead line, wherein the first metal layer comprises a metal or alloy which establishes a diffusion couple with the tin-based coating that promotes a bulk material deficiency in the tin-based coating and, thereby, an internal tensile stress in the tin-based coating; and
depositing the tin-based coating over the first metal layer to a thickness between about 0.5 μm and about 4.0 μm.

25. The method of claim 24 wherein depositing the tin-based coating over the first metal layer is to a thickness between about 0.5 μm and about 3.0 μm.

26. The method of claim 24 wherein the metal lead line onto which the first metal layer and tin-based coating are deposited constitutes a segment of a lead frame to be incorporated into the electronic package.

27. The method of claim 24 wherein the metal lead line onto which the first metal layer and tin-based coating are deposited constitutes a segment of a lead line extending out of the electronic package, and the electronic package is encapsulated.

28. The method of claim 24 wherein:
the depositing the first metal layer comprises depositing Ni or Ni alloy to a thickness between about 0.1 and about 20 μm.

29. An electronic component comprising the tin-based coating applied by the method of claim 1.

30. A metal lead line of an electronic package, wherein the lead line comprises the tin-based coating applied by the method of claim 12.

31. A metal lead line of an electronic package, wherein the lead line comprises the tin-based coating applied by the method of claim 22.

32. An electronic component of an electronic device comprising:
a metal surface adapted to be electrically connected by soldering during assembly of the electronic device;
a tin-based coating having a thickness between about 0.5 and about 2.5 μm over the metal surface; and
a first metal layer between the metal surface and the tin-based coating, wherein the first metal layer comprises a metal or alloy which establishes a diffusion couple with the tin-based coating that promotes a bulk material deficiency and, thereby, internal tensile stress in the tin-based coating.

33. The electronic component of claim 32 wherein the first metal layer is selected from the group consisting of nickel and nickel alloys.

34. The electronic component of claim 32 wherein the first metal layer material has a thickness between about 0.1 μm and about 20 μm.
